(19) United States
(12) Patent Application Publication
(10) Pub. No.: US 2019/0167541 A1
(43) Pub. Date: Jun. 6, 2019

(54) COSMETIC PRODUCT FOR THE LIPS IN THE FORM OF A WATER-IN-OIL EMULSION AND MAKEUP METHOD

(71) Applicant: LVMH RECHERCHE, Saint-Jean de Braye (FR)

(72) Inventors: Fabienne BROSSARD, Orleans (FR); Amandine PHILIPPE, Orleans (FR); Valérie DE LA POTERIE, Lailly en Val (FR)

(21) Appl. No.: 16/321,373

(22) PCT Filed: Jul. 27, 2017
(86) PCT No.: PCT/FR2017/052101

§ 371 Date: Jan. 28, 2019

(30) Foreign Application Priority Data
Jul. 28, 2016 (FR) 1657296

(51) Int. Cl. A61K 8/06; A61K 8/34; A61K 8/25; A61K 8/37; A61K 8/73; A61K 8/891; A61Q 1/06

(52) U.S. Cl. CPC A61K 8/064; A61K 8/345; A61K 8/342; A61K 8/25; A61K 8/375; A61K 8/731; A61K 2800/592; A61Q 1/06; A61K 2800/87; A61K 2800/43; A61K 2800/612; A61K 2800/651; A61K 8/891

(57) ABSTRACT

The invention relates to a water-in-oil emulsion for making up the lips that is very fluid while remaining stable. For example, the emulsion contains at least 30% by weight of a mixture of oils, at least 30% by weight of water, from 10% to 20% by weight of a volatile solvent, from 2% to 4% by weight of ethylcellulose, from 1.5% to 2.5% by weight of a hectorite modified with an organic compound, said mixture of oils comprising isostearyl isotearate, at least one ester of polyglycerol, and octyldodecanol.

The present invention relates to a cosmetic composition in the form of a water-in-oil emulsion containing a particular mixture of oils, and it relates more particularly to the field of makeup for lips.

The invention also relates to a method of cosmetically making up the lips, comprising topical application of this composition on the lips.

### PRIOR ART

Makeup products for the lips are cosmetic products in which gloss properties are looked for, unlike other cosmetic products, such as for example skin care products, where it is desired to obtain a film on the skin with a mat finish that is as discreet as possible.

The level of retention of a makeup product over time also needs to be much greater on the lips than for a cosmetic product applied to other regions of the body, insofar as the product is subjected to greater friction.

Finally, the comfort procured by the product also needs to be very great since the lips are much more sensitive than other parts of the body.

Thus, in order to be suitable for being put on sale, a makeup product for the lips needs to satisfy complex specifications including many criteria such as: the stability of the product over time; the comfort of the product while being applied to the lips and also over time once it has been applied; the gloss of the film that is deposited on the lips; and the retention of the film on the lips over time.

These criteria are difficult to satisfy simultaneously: for example, increasing the retention of the makeup over time or increasing the gloss of the film deposited on the lips leads very frequently to a reduction in the comfort of the product on the lips, which can give rise to the lips drying, to sensations of stickiness, or of the deposit feeling heavy on the lips.

The great majority of makeup products for the lips are formulated from fats and do not contain water. Lipsticks or lip glosses containing water may be in the form of solid or liquid emulsions containing an aqueous phase and an oil phase. Either the aqueous phase is dispersed in the oil phase (a water-in-oil emulsion), or else the oil phase is dispersed in the aqueous phase (an oil-in-water emulsion).

The emulsion of the invention is in the form of a liquid water-in-oil emulsion and it presents the feature of being very fluid compared with prior art emulsions in this category. The inventors have discovered, unexpectedly, how to formulate an emulsion that is stable over time and that contains a large quantity of oils and a large quantity of water while using small quantities of stabilizing agents, which are generally needed to guarantee the long-term stability of an emulsion system. The emulsion of the invention also presents the advantage of containing small quantities of retention agents, in particular phenyl silicone compounds that can generate sticky sensations. The inventors have found in particular that associating an ethylcellulose, a hectorite modified with an organic compound, and esters of a polygylcerol makes it possible to achieve such an object. Specifically, it is very difficult to reconcile the stability, the fluidity, and the texture of an emulsion in order to avoid it generating stickiness or discomfort on the lips.

Providing a water-in-oil emulsion of great fluidity for making up the lips presents numerous advantages: it makes it possible to obtain films on the lips that provide a sensation of freshness during application because of the presence of water, and also a sensation of lightness that is associated essentially with their small thickness. In spite of its fluidity, the emulsion of the invention makes it possible to obtain very good moisturizing and gloss properties. This is even more surprising in that the film deposited on the lips is thin.

One of the objects of the invention is thus to propose a product for application to the lips that provides lip makeup that is very moisturizing on application and over time without generating discomfort. Another object of the invention is to obtain a deposit of the product on the lips that is very thin and that leaves a sensation of being flexible, smooth, light, soft, non-greasy, and not sticky on the lips.

### GENERAL DESCRIPTION OF THE INVENTION

Below in this text, percentages are expressed by weight relative to the total weight of the emulsion of the invention, unless specified explicitly to the contrary.

At least one of the objects of the invention set out above is achieved by a water-in-oil emulsion having dynamic viscosity at 25° C. and at atmospheric pressure that lies preferably between 1500 millipascal seconds (mPa·s) and 10,000 mPa·s, the emulsion comprising an aqueous phase, oils, one or more volatile solvents, and coloring matter.

This emulsion may satisfy any of the following characteristics or a combination of more than one of these characteristics:
the aqueous phase represents between 25% and 45% by weight and comprises water, at least one glycol, and optionally at least one moisturizer;
the oils represent between 25% and 50% by weight and comprise at least one first oil selected from among polygylcerol esters, at least one second oil selected from fatty alcohol monoesters, and at least one third oil selected from C10-C26 fatty alcohols; and
the volatile solvent(s) represent(s) from 10% to 20%, preferably from 12% to 18% by weight.

Said emulsion may also contain from 0.5% to 10% by weight, preferably from 2% to 4% by weight of a soluble polymer or of a polymer dissolved in said third oil. Said emulsion may also contain from 0.05% to 5% by weight, preferably from 0.1% to 2% by weight of a gelling compound for gelling one of the oils or the mixture of oils mentioned above. The term “gelling compound” or “oil gelling compound” is used to designate a compound that increases the dynamic viscosity at 25° C. and at atmospheric pressure of the oil or of the oil mixture to which it is added at a content from 0.4% to 20% by weight relative to the weight of the oil or to the oil mixture concerned.

### DETAILED DESCRIPTION OF THE INVENTION

In one of its aspects, the invention provides a water-in-oil emulsion comprising:
from 25% to 45% by weight of an aqueous phase comprising water and at least one polyol;
from 25% to 50% by weight of a mixture of oils;
from 10% to 20% by weight of at least one volatile solvent;
from 0.5% to 10% by weight of a polymer dissolved in at least one of said oils or in the mixture of said oils;
from 0.05% to 5% of a gelling compound for gelling at least one of said oils or for gelling the mixture of said oils; and
coloring matter;
the percentages being expressed relative to the weight of the emulsion;
the emulsion being characterized in that the mixture of oils comprises, preferably in the majority, at least a first oil selected from the esters of a polyglycerol, at least one second oil selected from fatty acid monoesters, and at least one third oil selected from fatty alcohols.

In this aspect:
water may represent from 30% to 40% by weight of water, and the polyol may represent from 3% to 15% by weight;
the first oil may be a mixture comprising at least two preferably non-hydroxylated esters of polyglycerol and of C16-C20 fatty alcohol, and may represent from 1% to 15% by weight;
the second oil may be a non-hydroxylated monoester comprising at least one and preferably two saturated C16-C20 alkyl chains, and may represent from 1% to 15% by weight;
the third oil may be a C10-C26 saturated monoalcohol and may represent from 10% to 30% by weight;
the volatile solvent may comprise at least 80% by weight of a silicone solvent;
the gelling compound may be a hectorite modified with a quaternary alkylammonium chloride and may represent from 0.1% to 1% by weight; and
the coloring matter may represent from 0.1% to 5% by weight;
the percentages being expressed by weight relative to the weight of the emulsion.

This emulsion may present the advantage of being both very fluid and also stable. Its dynamic viscosity at 25° C. and at atmospheric pressure preferably lies between 1500 mPa·s and 10,000 mPa·s.

A person skilled in the art can use techniques forming part of that person's general knowledge to verify that the aqueous phase is dispersed in another phase containing said oils.

In another aspect, a water-in-oil emulsion of the invention having dynamic viscosity at 25° C. and at atmospheric pressure that lies preferably between 1500 mPa·s and 10,000 mPa·s, may comprise an aqueous phase, oils, one or more volatile solvents, and coloring matter, and may be characterized in that:
the aqueous phase represents between 25% and 45% by weight;
the oils represent between 25% and 50% by weight and comprise at least a first oil selected from among esters of a polygylcerol; and
the volatile solvent(s) represent from 10% to 20% by weight;
said emulsion also containing from 1% to 4% by weight of ethylcellulose, and from 0.05% to 5% by weight of a gelling compound for gelling one of said oils or the mixture of said oils;
the percentages being expressed by weight relative to the weight of the emulsion.

In another aspect of the invention, a water-in-oil emulsion contains:
from 30% to 40% by weight of water;
from 1% to 15% by weight of a non-hydroxyl monoester comprising at least one and preferably two saturated C16-C20 alkyl chains;
from 10% to 30% of a C10-C26 saturated monoalcohol;
from 0.1% to 10% by weight of at least one polymer dissolved in said monoalcohol;
from 10% to 20% by weight of a mixture of volatile solvents comprising at least 80% by weight of a silicone solvent;
from 1% to 15% by weight of a mixture comprising at least two, preferably non-hydroxyl, esters of polygylcerol and of C16-C20 fatty alcohol;
from 0.1% to 1% by weight of a hectorite modified with quaternary alkylammonium chloride; and
from 3% to 15% by weight of polyols;
the percentages being expressed by weight relative to the weight of the emulsion.

In the present application, the term “from . . . to . . . ” seeks to include the upper and lower bounds of the range of values, while the term “between . . . and . . . ” excludes the bounds of the range of values. Disclosing a range of values excluding its bounds amounts to disclosing an equivalent range of values including the bounds, and vice versa.

In the meaning of the present invention, an “oil” is a compound that is not soluble at 25° C. and at atmospheric pressure in the aqueous phase of the emulsion at the quantities at which the aqueous phase and the oil are used. A “liquid” compound is a molecule or a mixture of molecules having a melting point, a softening temperature, or a glass transition point that is less than or equal to 30° C., preferably less than or equal to 25° C., or even less than or equal to 20° C., the melting point corresponding to the maximum of the curve obtained by scanning differential calorimetry, or to the starting melting point of the curve obtained by scanning differential calorimetry. The term “solid” designates a compound that is not liquid.

The term “fatty acid” is used to mean a saturated, or unsaturated, linear or branched, carboxylic acid containing from 8 to 30 atoms of carbon, preferably from 10 to 24 atoms of carbon, more preferably from 12 to 22 and better from 16 to 20 atoms of carbon, optionally substituted by one or more hydroxyl groups. In a particular embodiment of the invention, the fatty acid(s) is/are linear, saturated, and not substituted by a hydroxyl group.

In a particular embodiment, the emulsion described in accordance with one of the above aspects contains at least 25% by weight of a mixture of oils, at least 25% by weight of water, from 10% to 20% by weight of a volatile silicone solvent, from 0.5% to 10% and preferably from 2% to 4% by weight of an ethylcellulose, from 0.05% to 5% by weight of a hectorite modified with an organic compound, said mixture of oils comprising isostearyl isotearate, at least one polyglycerol ester, and octyldodecanol.

In one of its above-described aspects, the emulsion of the invention is preferably a makeup product for the lips, i.e. a product having particular properties and that is not appropriate for other uses such as a skin care product (lotion, serum, cream), a skin makeup product (foundation), or an eyelash makeup product (mascara).

Finally, the last aspects of the invention relate to: i) a method of making up or caring for the lips that consists in applying one of the above-described emulsions on the lips; ii) a method of fabricating one of the above-described emulsions; and iii) a bottle containing one of the above-described emulsions.

Each of the above specified aspects may comprise or make use of any one of the ingredients described below.

The aqueous phase advantageously represents from 25% to 45% by weight, e.g. from 30% to 40% by weight relative to the weight of the emulsion. The aqueous phase may contain water, a polyol, and/or a sodium or magnesium salt, in particular sodium chloride.

A polyol may be incorporated in the aqueous phase and may include at least two hydroxyl groups with from 3 to 8 atoms of carbon. By way of example, the polyol may be selected from propylene glycol, pentylene glycol, caprylyl glycol, 1,3 propanediol, butylene glycol, and glycerol.

The emulsion of the invention may comprise from 3% to 15% by weight of polyol(s), in particular from 5% to 10% by weight of polyol(s) relative to the weight of the emulsion.

The emulsion of the invention may comprise at least a first oil selected from polyglycerol esters. The polyglycerol ester is advantageously selected from non-oxyalkylene esters of polyglycerol, e.g. non-oxyalkylene esters of polyglycerol-2.

The polyglycerol ester may have both an emollient function and also a function of stabilizing the aqueous phase dispersed in the fatty phase.

A polyglycerol ester may be selected from the esters that result from esterification of a polyglycerol with a polyricinoleic acid, referred to as polyglyceryl polyricinoleate. By way of example, mention may be made of polyglyceryl-3 polyricinoleates, polyglyceryl-5 polyricinoleates, polyglyceryl-6 polyricinoleates, and polyglyceryl-10 polyricinoleates (to use their INCI names). In a particular embodiment of the invention, polyglyceryl-6 polyricinoleate is selected. The emulsion of the invention may comprise from 1% to 3% by weight of a polyglyceryl polyricinoleate compound, in particular from 1.5% to 2% by weight relative to the weight of the emulsion.

The fatty acids are preferably selected from saturated fatty acids that are not substituted by a hydroxyl group and that comprise from 8 to 30 atoms of carbon and possibly at least one methyl branch. The fatty acids may be selected from oleic acid, lauric acid, palmitic acid, myristic acid, stearic acid, isostearic acid, linoleic acid, capric acid, behenic acid, or mixtures thereof.

Another polyglycerol ester may be selected from among the esters that result from esterification of a polyglycerol with a saturated fatty acid that is not substituted by a hydroxyl group, optionally comprising at least one methyl branch and possibly from 16 to 20 atoms of carbon, preferably isostearic acid. The polyglycerol esters contained in the emulsion are preferably selected from non-oxyalkylene esters of polyglycerol-2. By way of example, the polyglycerol ester is thus polyglyceryl-2 triisostearate, polyglyceryl-2 isostearate, or polyglyceryl-2 diiosostearate.

In a particular embodiment of the invention, the first oil is a mixture comprising at least a first ester selected from the esters of a polyglycerol and an aliphatic carboxylic acid having from 16 to 20 atoms of carbon, and at least one second ester selected from polyglyceryl polyricinoleates. The ester of polyglycerol and of C16-C20 fatty acid is selected in particular from polyglyceryl-2 triisostearate, polyglycerol-2 isostearate, and mixtures thereof.

Thus, the first oil may be a mixture of polyglyceryl-2 triisostearate, of polyglyceryl-2 isostearate, and of polyglyceryl-6 polyricinoleate.

The first oil preferably represents from 1% to 15% by weight relative to the weight of the emulsion, more precisely from 5% to 10% or from 6% to 8% by weight relative to the weight of the emulsion.

The emulsion of the invention may contain at least one second oil that is selected from fatty acid monoesters, the fatty acids being as defined above. By way of example, non-hydroxyl monoesters may be selected comprising at least one branched C16-C20 alkyl chain. Such monoesters may be selected from esters of a C16-C20 aliphatic carboxylic monoacid and a C16-C20 aliphatic monoalcohol, at least one of a monoacid or of the monoalcohol including at least one methyl or ethyl branch.

The monoesters preferably comprise a C18 branched alkyl chain.

Esters of isostearic acid are preferred. By way of example, the second oil may be selected from the following compounds: myristyl isostearate; butyl isostearate; isostearyl isostearate; stearyl isostearate; isocetyl isostearate; isopropyl isostearate; hydrogenated castor oil isostearate; and 2-ethyl-hexyl isostearate (to use their INCI names).

In a particular embodiment of the invention, the second oil is isostearyl isostearate.

The weight of all of the liquid esters contained in the emulsion of the invention is preferably greater than 30%, preferably greater than 35%, more preferably greater than 40% of the weight of all of the oils of said emulsion. In a particular embodiment, more than 90% of the ester oils contained in the emulsion of the invention are C16-C20 fatty acid esters, preferably isostearic acid esters. The emulsion of the invention may comprise between 10% and 20%, preferably between 14% and 18% by weight of oils having an ester function, relative to the weight of the emulsion. An additional ester oil may be an ester of 1,1,1-trimethylolpropane and of a fatty acid, e.g. trimethylolpropane triisostearate.

A third oil may be selected from C10-C26 aliphatic fatty alcohols, preferably from C10-C26 saturated linear or branched aliphatic monoalcohols, more preferably C10-C20 monoalcohols, such as 2-octyldodecanol.

The emulsion preferably comprises between 15% and 30%, preferably between 20% and 25% of the third oil.

The emulsion of the invention may incorporate a quantity of polymer that confers film-generating properties to a deposit of the emulsion on the lips. It is preferable to use a polymer that is solid at 25° C. and that is dissolved in an oil so that the polymer is dissolved in the oil when it is introduced into the emulsion.

The polymer is soluble or dissolved in at least one of the above-described oils or in the mixture of above-described oils. The polymer is said to be soluble when it is dissolved at a concentration by weight lying between 5% and 40% by weight relative to the weight of said oil or of said mixture of oils.

Prior to being introduced into the emulsion of the invention, the polymer may be selected from polymers that are soluble at 25° C. in the third oil when its concentration in the third oil lies between 10% and 30% by weight. The polymer is preferably a hydrocarbon. The term “hydrocarbon” is used to mean a polymer that does not include silicon.

Such a polymer, prior to being introduced into an oil, may be solid at 25° C., and may be selected from ethylcelluloses, plant resins, and copolymers of vinylpyrrolidone.

Plant resins, in the meaning of the invention, cover several different compounds:
a substance secreted by certain plants and then collected without subsequent chemical transformation, referred to as a resinous exudate, e.g. a resinous exudate obtained in particular by tapping a tree trunk;
a molecule extracted from a resinous exudate, or a mixture of such molecules; and
the products of chemically reacting a resinous exudate or a molecule it contains.

When plant resins are harvested from plants without chemical transformation, they may be either in liquid form or else in solid form after being dried by eliminating any water they might contain. Plant resins are generally liquids that are very viscous and that dry more or less quickly when they are placed in contact with air.

By way of example, a derivative of a resinous exudate is an ester of glycerol and of rosin (also known as glyceryl rosinate or monoester of glycerol and of a mixture of long-chain fatty acids derived from rosin, or glyceryl monorosinate, CAS 65997-13-9, CAS 8050-31-5). Rosin (C₁₅H₂₀O₆; CAS 85026-55-7) is an aromatic acid having a glucopyranoside motif that is extracted from colophane, which is obtained after distilling and drying an exudate harvested from resinous trees such as pine trees.

It is also possible to use a resin coming from the Shorea Robusta tree (INCI name: Shorea Robusta Resin), also referred to as Damar gum, which is a resinous exudate that is harvested from the tree of genus Shorea and of species Robusta.

In a particular embodiment of the invention, the polymer is an ethylcellulose.

The degree to which hydroxyl groups are substituted by an ethylcellulose anhydroglucose motif preferably lies from 2.0 to 3.0 (which may correspond to a percentage by weight of ethyoxyl groups lying between 45.0% and 52.5% relative to the weight of the substituted polymer, depending on the grade used).

The mean molecular weight of the ethylcellulose is preferably selected so that the viscosity of a solution at 5% by weight in a mixture of 80 parts toluene and 20 parts ethanol at 25° C. goes from 1 mPa·s to 400 mPa·s, preferably from 3 mPa·s to 250 mPa·s, e.g. from 5 mPa·s to 20 mPa·s.

In a particular embodiment of the invention, the ethylcellulose is dissolved in an oil or in the mixture of oils of the emulsion of the invention, and may correspond to the grade sold under the trade names Ethocel® Standard 7 Premium, Ethocel® 100, or Ethocel® 200 (Dow Chemical Co.), or under the trade name Aqualon® NC (Ashland).

Among copolymers of vinylpyrrolidone, mention may be made of VP/eicosene and VP/hexadecene.

By way of example, the quantity of copolymer goes from 0.1% to 10% by weight, preferably from 1% to 5%, or even from 2% to 4% relative to the weight of the emulsion. The inventors have observed that at a content greater than 10%, the emulsion when spread to a thickness of one to a few hundreds of microns is thicker and stickier. At a content of less than 0.1%, the gloss and moisturizing properties of the emulsion when spread on the lips are not sufficient.

In a particular embodiment, use is made of a mixture of a polymer, e.g. such as ethylcellulose, and of a hectorite modified by an organic compound, such as for example a tetra-alkylammonium salt, e.g. a hectorite as described below.

It is possible to incorporate in the emulsion of the invention one or more gelling compounds for gelling one of the oils of the emulsion of the invention, or for gelling the mixture of oils contained in said emulsion. It is preferable to use from 0.05% to 5%, preferably from 0.1% to 2%, e.g. from 0.5% to 1% by weight of gelling compound(s).

As examples of gelling compounds suitable for use in the ambit of the invention, mention may be made of modified natural micas such as fluorosilicates of aluminum, of magnesium, and of potassium; the esters of dextrin and of fatty acid such as dextrin palmitate or dextrin myristate; tri-esters of C8-C30 fatty acids and of mono- or poly-glyceryl such as glyceryl tri(hydroxystearate) (INC name: trihydroxystearin); organomodified clays, which are clays treated by compounds selected in particular from quaternary amines, tertiary amines; and pyrogenic (fumed) silicas surface treated with a silicone compound.

The emulsion of the invention may contain from 0.1% to 1% by weight of a hectorite modified with quaternary alkylammonium chloride, preferably an ammonium substituted by at least one, preferably at least two, alkyls having from 14 to 20 atoms of carbon. The alkyl may be stearyl. Mention is made of the compound having the INC name disteardimonium hectorite, in which the ammonium has two methyls and two stearyls.

The emulsion of the invention advantageously contains the mixture of an ethylcellulose and of a hectorite modified with a quaternary alkylammonium chloride, as described above.

The volatile solvent(s) optionally used in the emulsion is/are preferably constituted by more than 65% by weight, preferably by more than 75%, or even more than 90% by weight of a silicone compound.

The volatile solvent may be selected in particular from silicone oils such as dimethicones having viscosity from 0.5 centistokes (cSt) to 6 cSt, octamethyl cyclotetrasiloxane, decamethyl cyclopentasiloxane, dodecamethyl cyclohexasiloxane, heptamethyl hexyltrisiloxane, heptamethyloctyl trisoloxane, hexamethyl disiloxane, octamethyl trisoloxane, decamethyl tetrasiloxane, dodecamethyl pentasiloxane, and mixtures thereof. In a particular embodiment of the invention, the volatile solvent is decamethyl cyclopentasiloxane.

The volatile solvent may also be a C1-C6 monoalcohol such as ethanol, or a hydrocarbon such as isododecane, isodecane, isohexadecane, n-dodecane (C12), and n-tetradecane (C14), and a mixture of undecane and tridecane.

The volatile solvent(s) present in the emulsion of the invention may represent from 5% to 20%, e.g. from 10% to 20%, preferably from 12% to 18% by weight relative to the weight of the emulsion.

The emulsion of the invention preferably contains less than 5% by weight of volatile alcohols and/or of volatile hydrocarbons, such as ethanol or isododecane, which might be found to be drying on the lips and can reduce the comfort and the moisturization that are looked for in the ambit of the invention.

The inventors have observed that volatile solvents such as isododecane and ethanol can reduce the moisturization of the lips provided by the emulsion, when they are used in certain proportions.

In a particular embodiment, the volatile solvent(s) contained in the emulsion is/are constituted by a cyclomethicone, preferably decamethyl cyclopentasiloxane.

The emulsion of the invention may contain one or more molecules and/or one or more plant extracts presenting moisturizing properties, such as glycols, in particular glycerol or natural polyols, or any other moisturizing agent known to the person skilled in the art.

The emulsion of the invention contains coloring matter that is selected from pigments (insoluble in the oils and in the aqueous phase) and dyes (soluble in the oils or in the aqueous phase).

Among mineral pigments, mention may be made by way of example of titanium dioxide, preferably that has been surface treated; black, yellow, red, and brown iron oxides; manganese violet; ultramarine blue; chromium oxide; hydrated chromium oxide; and ferric blue.

Among organic pigments, mention may be made by way of example of the following pigments: D & C Red No. 19; D & C Red No. 9; D & C Red No. 21; D & C Orange No. 4; D & C Orange No. 5; D & C Red No. 27; D & C Red No. 13; D & C Red No. 7; D & C Red No. 6; D & C Yellow No. 5; D & C Red No. 36; D & C Orange No. 10; D & C Yellow No. 6; D & C Red No. 30; D & C Red No. 3; carbon black, and lakes based on cochenille carmine.

Pearlescent pigments may be selected in particular from white pearlescent pigments, such as mica covered in titanium oxide, bismuth oxychloride; and colored pearlescent pigments such as titanium mica with iron oxides, titanium mica with ferric blue or chromium oxide, titanium mica with an organic pigment of the above-specified type, and also pigments based on bismuth oxychloride.

Among dyes, mention may be made of caramel, Yellow 5, Acid Blue 9/Blue 1, Green 5, Green 3/Fast Green FCF 3, Orange 4, Red 4/Food Red 1, Yellow 6, Acid Red 33/Food Red 12, Red 40, cochenille carmine (C1 15850, C1 75470), Ext. Violet 2, Red 6-7, Ferrie Ferrocyanide, Ultramarines, Acid Yellow 3/Yellow 10, Acid Blue 3, Yellow 10. By way of example, liposoluble dyes are Soudan red, D & C Red 17, D & C Green 6, beta carotene, soybean oil, Soudan brown, D & C Yellow 11, D & C Violet 2, D & C Orange 5, quinolene yellow, annatto.

By way of example, the emulsion of the invention contains from 0.01% to 10% by weight, e.g. from 0.1% to 1% by weight of coloring matter, and from 0.2% to 0.8% by weight relative to the total weight of the emulsion in a particular embodiment.

Other than the above-described ingredients, the emulsion of the invention includes at least one cosmetically or dermatologically acceptable excipient that may be selected from fragrances, phospholipids, electrolytes, sweeteners for masking the bitterness of certain compounds in the emulsion when it is applied to the lips, pH adjusters, and preservatives. A particular phospholipid is non-hydrogenated lecithin extracted from soybean comprising 23% by weight of phosphatidylcholine and 97% by weight of phospholipids and glycolipids.

The moisturizing properties of the emulsion of the invention can be measured by corneometry. Corneometry measures variation in the capacitance of the skin over time. By using a probe made up of two metal electrodes (made of gold) in the form of combs, a thin insulating layer lies between the electrodes at the end of the probe. An electric field is created at the surface of the skin. The capacitance of the electrode-and-skin system depends on the dielectric constant of the skin that is in contact with the probe, such that the more the skin is moist, the greater the dielectric constant. The moisturizing effect of the emulsion at time T can be expressed in the form of a percentage increase between the capacitance measured at the time T on a non-treated zone of the skin and the capacitance measured at the time T on a zone of the skin that has had the emulsion of the invention applied thereto.

The moisturizing power of an embodiment of the invention, as measured by corneometry, and in particular using the above-described protocol, may be greater than 60%, preferably greater than 65%, 6 hours (h) after it was applied.

The viscosity of the emulsion at 25° C. and at atmospheric pressure preferably lies between 1500 mPa·s and 10,000 mPa·s, is preferably less than 8000 mPa·s, 6000 mPa·s, or 5000 mPa·s, and more preferably lies between 2500 mPa·s and 4000 mPa·s.

This viscosity can be measured with a Rheolab QC (Anton Paar) viscosity meter using Rheoplus software with a moving body and a measurement time that are appropriate, e.g. under the following conditions:

| | Speed of rotation | Measurement |
|---|---|---|
| Shapes | (rpm) | time (min) |
| Coaxial cylinder: CC27 | 200 | 3 |
| 4 blades: ST22-4V | 100 | 3 |
| Fins: ST24-2D-2V-2V | 50 | 3 |
| Anchor: ST22-2V | 10 | 7 |

Prior to taking the measurement, the emulsion of the invention is placed in a 120 milliliter (mL) pot (Ref.: 102171001, Kola Rond VT3 M120 Blanc Pharm) in a stove at 25° C. for a minimum of 12 h. Once the moving body has been plunged into the pot, the level of the composition should reach the neck of the pot.

It is verified that the moving body is properly chosen by measuring the percentage deviation of the measurements that are taken every 6 seconds (s). With this problem, the value for the viscosity of the emulsion is equal to the mean of the last fifteen measurements taken with the apparatus during the above-specified measurement time.

A wet film of an emulsion of the invention advantageously presents thickness measured at 25° C. that is less than or equal to 65 micrometers (μm), preferably less than or equal to 60 μm when the emulsion is deposited on a glass plate using an automatic spreader and a bar having a thickness of 100 μm.

The thickness of a deposit of the emulsion can be evaluated by depositing the product on a glass plate using an automatic spreader and a bar having a thickness of 100 μm. The spread deposits may optionally be placed for 30 minutes (min) to 2 h in a hygrometrically controlled thermal enclosure at 25° C. and 50% relative humidity.

The thickness may be measured immediately after spreading or else on leaving the thermal enclosure, using any method known to the person skilled in the art.

An emulsion of the invention is advantageously stable in that its phases do not separate over time: the aqueous phase and the oils do not separate so as to form two distinct phases that can be distinguished by visual examination. Stability can thus be verified after the emulsion has been stored in a stove at 50° C., 45° C., or 4° C. for a period of 1 month or 2 months.

An emulsion in accordance with any of the aspects of the invention may satisfy any of the following characteristics or a combination of a plurality of these characteristics:
it may comprise less than 0.2% by weight, preferably less than 0.15% by weight, more preferably not have any aqueous gelling agent (i.e. a compound that increases the viscosity of water by more than 5% at 25° C. and at atmospheric pressure when it is soluble or dispersible in water), without that harming the stability of the emulsion. Under such circumstances, the resulting product is less sticky, more fluid, and more slippery on application. Aqueous gelling agents that may be mentioned comprise hydroxyethylcellulose, carboxylvinyl polymers (INCI name carbomer), polysaccharides such as xanthane gums, guar gums, carrageenans, hydroxymethylcelluloses, and mixtures thereof. The emulsion of the invention advantageously does not have any of the above-mentioned gelling agents,
it may comprise less than 1% by weight, preferably less than 0.5%, or even have no fatty compound(s) that is/are solid or include a solid phase, such as waxes or pasty compounds. Solid fatty compounds that may be mentioned in particular include stearic acid, a polyethylene wax, a synthetic wax, a paraffin wax, or a microcrystalline wax, cetyl alcohol, stearyl alcohol, behenyl alcohol, and glyceryl stearate. Specifically, these compounds may increase the thickness of the film of composition deposited on the lips and/or increase the sticky sensation that the film can generate,
it may comprise less than 6% by weight, preferably less than 1% by weight, preferably not have any of the following compounds: partially or totally cross-linked organopolysiloxane elastomers, copolymers of polystyrene and copoly(ethylene propylene) and of polystyrene and copoly(ethylene butylene); optionally hydrogenated polyisobutenes. Specifically, these compounds may leave a greasy and/or sticky sensation on the lips, which the invention seeks specifically to avoid,
it may comprise less than 5%, preferably less than 1% by weight of a non-volatile solid or liquid silicone compound, or indeed not contain any such compound; among silicone compounds that may be mentioned are silicone resins, silicone elastomers, silicone surfactants, silicone gums, or phenyl silicone oils. The inventors have found in particular that the presence in the emulsion of the invention of silicone compounds such as a phenylpropyl dimethylsiloxy silicate resin or a cetyl dimethicone copolyol leads to films being deposited on the lips that are perceived by users as being greasier, thicker (and thus more perceptible), and less comfortable over time. The emulsion of the invention advantageously contains less than 1%, preferably 0% by weight of non-volatile phenyl silicone compounds. In spite of the very low content or even absence of silicone resin compounds, of polysaccharides, or of phenyl dimethicones, the care and makeup properties of the products of the invention are retained in satisfactory manner, in particular moisturizing and gloss retention,
it may comprise less than 0.5% by weight of organic sunscreens, since they reduce the sensory qualities of the emulsion,
it may contain less than 8% by weight, preferably less than 5% by weight, preferably have no ester oil(s) selected from triglycerides derived from plant matter (e.g. castor oil) and pentaerythrityl tetraisostearate,
it may comprise less than 2% by weight, preferably have no sorbitan ester(s), or sucrose ester(s), such as sucrose cocoate or sorbitan stearate.

The invention also provides a method of making up the lips that consists in applying one of the above-described emulsions on the lips. All of the above-described characteristics concerning those emulsions apply to the makeup method of the invention.

The invention also provides a bottle provided with applicator means and containing the above-described emulsion. The emulsion of the invention is advantageously packaged in a bottle (or a squeeze pouch) having applicator means and a cap (or stopper). The applicator means may be a brush or a cellular foam, and it may advantageously be secured to the cap. The brush is preferably flat and its tip may be straight or rounded. The bottle may be in the shape of a cylinder, a tube, or a rectangular box. The applicator may have various shapes—cylindrical, oblong, or flat, for example—and it may optionally be chamfered so as to improve the accuracy with which the emulsion is applied to the lips.

Finally, the invention provides a method of preparing an emulsion as described above. A particular implementation of this method comprises at least three steps. In a first step, the second and third oils are mixed together, and then the polymer is dissolved in that mixture at a temperature higher than or equal to 60° C. In a second step, the temperature of the mixture obtained at the end of the first step is reduced to a value lying between 30° C. and 60° C., and then the other oils, the oil gelling agents, and the volatile solvent(s) are added, and then the temperature of the mixture is lowered to ambient temperature. Finally, in a third step, all of the ingredients of the aqueous phase are mixed together and then the aqueous phase is poured into the mixture obtained in the second step. In a fourth step, pigments may be added to the resulting emulsion. The mixing of all of the ingredients is advantageously performed under stirring and at atmospheric pressure.

The invention is illustrated in greater detail by the following examples.

### Examples

Two emulsions were prepared having the following compositions.

| Ingredients | | |
|---|---|---|
| INCI NAME (in upper case) | | |
| and/or function and/or | | Comparative |
| chemical name (in lower | Example 1 | Example 2 |
| case) | % by weight | |
| ISOSTEARYL ISOTEARATE | 9.0 | |
| OCTYLDODECANOL | 22.0 | 21.0 |
| Polymer soluble in | 3.0 | 5.0 |
| octyldodecanol | | |
| (ETHYLCELLULOSE Ethocel ® 7 | | |
| FP) | | |
| Esters of polyglycerol | 6.9 | |
| Volatile solvent | 15.8 | 16.7 |
| PENTAERYTHRITYL | | 8.1 |
| TETRAISOSTEARATE | | |
| HYDROGENATED POLYISOBUTENE | | 6.7 |
| Oil gelling agent | 0.6 | 0.8 |
| LECITHIN | 0.5 | 1 |
| SORBITAN SESQUIOLEATE | | 3.0 |
| Preservative | qs | qs |
| Anti-oxidant | qs | qs |
| WATER | qsp 100 | qsp 100 |
| GLYCERIN | 5.0 | 5.0 |
| CHONDRUS CRISPUS | | 0.2 |
| (CARRAGEENAN) | | |
| SODIUM CHLORIDE | 0.2 | 0.2 |
| ALCOHOL | | 1.4 |
| PENTYLENE GYLCOL | 3.0 | 2.0 |
| Pigments | 0.5 | 1.5 |

The emulsion of Example 1 of the invention was fabricated by mixing the ingredients in the order in which they appear in the above table.

In a first step, the second and third oils were mixed together and then the ethylcellulose was dissolved in that mixture at a temperature of 90° C. In a second step, the temperature of the mixture obtained at the end of the first step was lowered to 50° C., and then the other oils, the oil gelling agents, and the volatile solvents were added, and then the temperature of the mixture was lowered to 25° C. Finally, in a third step, all of the ingredients of the aqueous phase were mixed together and then the aqueous phase was poured into the mixture obtained in the second step. The pigments were added to the resulting emulsion in a fourth step. The mixing of all of the ingredients was performed under stirring and under atmospheric pressure.

—Stability Study

The emulsions were placed in a stove at 50° C. for 1 month.

Result: the product of Example 1 of the invention was stable, while the product of the comparative Example 2 was not.

—Viscosity Measurement

The viscosity of the emulsion in Example 1 of the invention and that of a lip gloss corresponding to a prior art product available on the market were measured using a Rheolab QC (Anton Paar) viscosity meter having Rheoplus software.

The viscosity of Example 1 of the invention was measured with the fin moving body rotating at 50 revolutions per minute (rpm) for 3 min, while the viscosity of the commercial product (Mintel Report No. 2401385) was measured using the Anchor moving model rotating at 10 rpm for 7 min.

Before the measurement, each composition was poured into a 120 mL pot (Ref.: 102171001, Kola Rond VT3 M120 Blanc Pharm) and then placed in a stove at 25° C. for a minimum of 12 h. Once the moving body was dipped into the pot, the level of the composition reached the neck of the pot.

The value of the viscosity of the emulsion is equal to the mean of the last fifteen measurements taken by the apparatus during the above-specified measurement time.

Result: the viscosity of Example 1 of the invention lay between 2500 mPa·s and 4000 mPa·s, and the viscosity of a prior art commercial product in the form of a water-in-oil emulsion (Mintel Report No. 2401385) lay between 10,000 mPa·s and 22,000 mPa·s. The emulsion of the invention was much more fluid than the emulsion of the prior art.

—Moisturizing Power

The moisturizing power of Example 1 of the invention was measured using a CM 825 (Courage and Khazaka) corneometer on ten Caucasian volunteers, making an application on the forearm (T=0).

Two zones of skin occupying 25 square centimeters (cm²) were selected randomly distributed over the inside face of the forearm of each volunteer. The emulsion was applied to one of the two specified zones at 2 milligrams per square centimeter (mg/cm²) in in-use manner, the untreated second zone serving as a control.

The moisturizing effect of the emulsion at time T=X h is equal to the percentage increase between the capacitance measured at time T=X h on the untreated zone of the skin of the forearm and the capacitance measured at time T=X h on a zone of the skin of the forearm on which the emulsion of Example 1 of the invention had been applied and after removing the residual film.

The same protocol was used for measuring the moisturizing power of a commercial product in the form of a water-in-oil emulsion (Mintel Report No. 2401385).

Result: the moisturizing power of Example 1 of the invention at T=6 h was +68%. The moisturizing power of the prior art product at T=8 h was +57%. In both cases, the variations that were observed were significant (p<0.01).

The emulsion of the invention presents moisturizing power that is clearly better than that of the prior art emulsion.

—Self Evaluations In Vivo

The product of Example 1 of the invention and a prior art commercial product (Mintel Report No. 2401385) were evaluated in independent manner by two different panels following the same protocol.

Panel for evaluating Example 1 of the invention: 32 volunteer women of Caucasian type aged 21 to 64 (average age 47). Application to the lips once per day for one week, with retouching during the day.

Panel for evaluating a prior art commercial product (Mintel Report No. 2401385): 32 volunteer women of Caucasian type aged 26 to 70. Application to the lips twice per day for four consecutive weeks.

Each woman of the panel herself evaluated the effects and the properties of the product by answering a questionnaire at the end of the test period. The volunteers answered a questionnaire by marking: “agree”, “somewhat agree”, “somewhat disagree”, “strongly disagree”. For each item, the satisfaction percentage (the number of “agree” and “somewhat agree” answers) was calculated.

Results: the Results obtained are set out in the table below.

| | | Prior art |
|---|---|---|
| | | commercial |
| Evaluated property | Example 1 | product |
| The texture is not sticky | 88 | 59 |
| The film is thin and light | 91 | 69 |
| It is not felt on the lips | 94 | 78 |
| (it is forgotten) | | |
| The product procures a | 84 | 72 |
| sensation of freshness on | | |
| application | | |
| The texture is agreeable to | 94 | 84 |
| wear | | |
| The product is applied | 91 | 81 |
| easily | | |
| The film is neither too thin | 84 | 75 |
| nor too thick | | |
| The lips are supple and soft | 94 | 84 |

All of the results were statistically significant.

The makeup result obtained with the emulsion of Example 1 of the invention was felt by the panel of volunteers to be thinner, more comfortable, very agreeable to wear, and less sticky.

—Measuring Stickiness

The emulsion of Example 1 of the invention was deposited on a glass plate using an automatic spreader and a bar having a 100 μm gap. The spreads of emulsion were deposited for 1 h in a thermal enclosure with controlled hygrometry, at 25° C. and 50% humidity.

Using a TAXT Plus (Stable Micro Systems) texture analyzer, fitted with a 5 kilogram (kg) force sensor, a constant pressure was applied for a determined length of time by means of a stainless steel cylinder having a diameter of 5 mm, and then the tool was removed at constant speed. During the removal stage, the emulsion exerts a suction force on the tool, which is correlated with the stickiness of the emulsion.

Five measurements were taken for each spread. Between two measurements, the cylinder was always cleaned using ethanol. Measurements were never taken at the same location on the sample. The stickiness of the emulsion is equivalent to the work done by the separation force as measured during the stage of withdrawing the tool. It corresponds to the integral of the curve along the time axis. This work is expressed positively as joules per square meter (J/m²).

Measurement Parameters:
1) approach speed: 0.1 millimeters per second (mm/s);
2) speed from detection of contact: 0.1 mm/s;
3) force/pressure: 0.197 newtons
   (N)/0.01 megapascals (MPa);
4) contact time: 3 s;
5) withdrawal speed: 0.1 mm/s.

The above-described protocol was reproduced using a prior art commercial product (Mintel Report No. 1716698).

Result: the stickiness of the emulsion of Example 1 of the invention was equal to 1.43±0.06 J/m². The value of the stickiness of the prior art product was equal to 1.79±0.31 J/m².

—Measuring the Thickness of a Film of Composition

A spread of the emulsion of Example 1 of the invention or a spread of a prior art commercial product (Mintel Report No. 2401385) was deposited on a glass plate using an automatic spreader with a 100 μm thick bar.

Thereafter, the thickness of those moist deposits was measured after leaving them for one hour in an enclosure at 25° C. and 50% relative humidity.

Result: the thickness of a film of the emulsion of Example 1 was 54.3 μm (standard deviation=4.2), and the thickness of the commercial product was 69.2 μm (standard deviation=1.5).

A spread of the emulsion of the invention was much thinner than a spread of another emulsion of the prior art, when the two products were spread using the same protocol.

1. A water-in-oil emulsion comprising:
from 25% to 45% by weight of an aqueous phase comprising water and at least one polyol;
from 25% to 50% by weight of a mixture of oils;
from 10% to 20% by weight of at least one volatile solvent;
from 0.5% to 10% by weight of a polymer dissolved in at least one of said oils or in the mixture of said oils;
from 0.05% to 5% of a gelling compound for gelling at least one of said oils or for gelling the mixture of said oils; and
coloring matter;
the percentages being expressed relative to the weight of the emulsion;
the emulsion being characterized in that the mixture of oils comprises at least one first oil selected from esters of a polyglycerol, at least one second oil selected from fatty acid monoesters, and at least one third oil selected from fatty alcohols.

2. An emulsion according to claim 1, characterized in that the aqueous phase represents from 30% to 40% by weight relative to the weight of the emulsion.

3. An emulsion according to claim 1, characterized in that the first oil is a mixture comprising a first ester selected from esters of a polyglycerol and of an aliphatic carboxylic acid comprising from 16 to 20 atoms of carbon, and a second ester selected from polyglyceryl polyricinoleates.

4. An emulsion according to claim 3, characterized in that the first ester is selected from the group consisting of polyglyceryl-2 triisostearate, polyglycerol-2 isostearate, and mixtures thereof.

5. An emulsion according to claim 1, characterized in that the second oil is a non-hydroxylated monoester having at least one C16-C20 aklyl chain selected from the group consisting of isostearyl isostearate; isocetyl isostearate; isopropyl isostearate; 2-ethyl-hexyl isostearate.

6. An emulsion according to claim 1, characterized in that it comprises between 10% and 20%, by weight relative to the weight of the emulsion of oils having an ester function.

7. An emulsion according to claim 1, characterized in that the volatile solvent is selected from silicone oils.

8. An emulsion according to claim 1, characterized in that the polymer is dissolved in the third oil.

9. An emulsion according to claim 1, characterized in that the third oil is octyldodecanol and the polymer is ethylcellulose.

10. An emulsion according to claim 1, characterized in that the oil gelling compound is a hectorite modified with a quaternary alkylammonium chloride and represents from 0.1% to 1% by weight relative to the weight of the emulsion.

11. An emulsion according to claim 1, characterized in that:
water represents from 30% to 40% by weight, and the polyol represents from 3% to 15% by weight;
the first oil is a mixture comprising at least two non-hydroxylated esters of polyglycerol and of C16-C20 fatty acid, and represents from 1% to 15% by weight;
the second oil is a non-hydroxylated monoester comprising at least two saturated C16-C20 alkyl chains, and represents from 1% to 15% by weight;
the third oil is a C10-C26 saturated monoalcohol and represents from 10% to 30% by weight;
the volatile solvent comprises at least 80% by weight of a silicone solvent;
the gelling compound is a hectorite modified with a quaternary alkylammonium chloride and represents from 0.1% to 1% by weight; and
the coloring matter represents from 0.1% to 5% by weight;
the percentages being expressed by weight relative to the weight of the emulsion.

12. A method of making up the lips that consists in applying an emulsion according to claim 1 to the lips.

13. A bottle provided with applicator means and a cap, which bottle contains an emulsion according to claim 1, said applicator means being a brush or a cellular foam.
